(19) United States
(12) Patent Application Publication
(10) Pub. No.: US 2023/0099696 A1
(43) Pub. Date: Mar. 30, 2023

(54) NANO-COMPOSITE MICROPARTICLES OF POLYMYXIN

(71) Applicant: Purdue Research Foundation, West Lafayette, IN (US)

(72) Inventors: Qi ZHOU, West Lafayette, IN (US); Chune ZHU, Guangzhou, Guangdong (CN)

(21) Appl. No.: 17/908,492

(22) PCT Filed: Mar. 5, 2021
(86) PCT No.: PCT/US2021/020993

§ 371 Date: Aug. 31, 2022

Related U.S. Application Data
(60) Provisional application No. 62/987,918, filed on Mar. 11, 2020.

(51) Int. Cl. A61K 38/12; A61K 9/16; A61K 9/19

(52) U.S. Cl. CPC A61K 38/12; A61K 9/16; A61K 9/19

(57) ABSTRACT

The present disclosure generally relates to a process for manufacturing a microparticle composite or a dry powder inhaler composition of polymyxin and CFTR activator or CFTR potentiator comprising the steps preparing a suspension of nanoparticles (NPs) of CFTR activator or CFTR potentiator or their combinations; mixing the suspension of NPs with a solution of polymyxin and a solution of leucine, then followed by drying to afford said micro-particle composite or dry powder inhaler composition. Pharmaceutical compositions and methods for treating a patient of cystic fibrosis (CF) and/or a lung infection are within the scope of this invention.

### GOVERNMENT SUPPORT CLAUSE

This invention was made with government support under AI132681 awarded by the National Institutes of Health. The government has certain rights in the invention.

### CROSS REFERENCE TO RELATED APPLICATIONS

This present patent application relates to and claims the priority benefit of U.S. Provisional Application Serial No. 62/987,918, filed Mar. 11, 2020, the content of which is hereby incorporated herein by reference in its entirety.

### TECHNICAL FIELD

The present application generally relates to a process for manufacturing a microparticle composite or a dry powder inhaler composition comprising colistin (Col), Ivacaftor (Iva), bovine serum albumin (BSA), and L-leucine for an effective co-delivery of Col and Iva. Pharmaceutical compositions and methods for treating a patient of cystic fibrosis (CF) and/or a lung infection are within the scope of this invention.

### BACKGROUND

This section introduces aspects that may help facilitate a better understanding of the disclosure. Accordingly, these statements are to be read in this light and are not to be understood as admissions about what is or is not prior art.

Cystic Fibrosis (CF) is an autosomal recessive disease triggered by mutations in the gene encoding of CF transmembrane conductance regulator (CFTR) protein (Garbuzenko et al., 2019). Many organs such as lungs, pancreas, kidneys, and intestines are affected by CFTR mutations (Porsio et al., 2018). As for the lung, mutations in the CFTR protein results in production of thick and sticky mucus, which lead to airways obstruction, severe repeated infection, inflammation and eventually lung failure (McColley et al., 2019). Recent studies have identified that the main bacteria species of lung infections in CF patients containing Staphylococcus aureus (SA) and/or Pseudomonas aeruginosa (PA). The lung infections by SA and PA play a key role in morbidity and mortality in CF patients, which are one of the therapeutic goals for CF (Katherine Fesen, 2019).

Ivacaftor (Iva), approved by Food and Drug Administration (FDA) in 2012 for the treatment of CF, is a CFTR “potentiator”. It extends opening time of the ion channel formed by the CFTR protein (Hamilton et al., 2018; Hubert et al., 2017). Due to the quinoline ring structure, Iva showed antimicrobial activity against SA with an MIC of 8~32 mg/L (Reznikov et al., 2014; Thakare et al., 2017). In addition, recent research reported that Iva has a significant antimicrobial activity against PA when combined with polymyxin B (Schneider et al., 2016).

Colistin (Col) belongs to the polymyxin family, which is a polycationic cyclic peptide that has potent bactericidal activity against the Gram-negative bacteria including PA, Acinetobacter baumannii and Klebsiella pneumoniae (Liu et al., 2018). The antibacterial mechanism of Col is known to be the strong electrostatic interactions between the positively charged drug molecule and the negatively charged bacterial membranes (Moffatt et al., 2010). Recently, due to the rapid resistance development against the first-line antibiotics, Col has been increasingly used as the last-line antibiotic against multidrug-resistant Gram-negative bacteria such as PA in CF patients (d’Angelo et al., 2015; Liu et al., 2018). However, previous studies showed that the efficacy of parenteral Col for lung infections in CF patients are very disappointing due to the low delivery efficiency of parenteral Col to the infections sites at the surface of deep lungs (Velkov et al., 2015). Simply increasing the dose of parental Col is not an option due to dose-limiting severe nephrotoxicity (Garonzik et al., 2011). In the past decade, inhaled Col becomes a complimentary therapy for lung infections in CF patients (Velkov et al., 2015). There are still spaces for further improvement for the treatment options for Cystic Fibrosis (CF) with Ivacaftor (Iva) in clinic.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIG. 1A is the SEM image of Iva-BSA-NPs with the scale bar of 500 nm;

FIG. 1B shows the PXRD patterns of Iva, PM of Iva and BSA, Iva-BSA-NPs and BSA;

FIG. 1C demonstrates the formation mechanism of Iva-BSA-NPs

FIG. 2 shows the particle size distributions of powder formulations with different solid contents.

FIG. 3A shows the PXRD patterns of formulations, raw materials and PM;

FIG. 3B shows the PXRD patterns of above materials exclude L-leucine and PM.

FIG. 4 shows representative SEM images for DPI formulations at two different magnifications for the various formulations F1, F2, F3, F4, F5, and F6, respectively.

FIG. 5 depicts the in-vitro dissolution profiles of F2 and jet-milled PM (n = 3)

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

As used herein, the following terms and phrases shall have the meanings set forth below. Unless defined otherwise, all technical and scientific terms used herein have the same meaning as commonly understood to one of ordinary skill in the art.

As it is disclosed herein, a nanoparticle refers to a particles having the size ranging from about 1 nm to about 999 nm. A microparticle refers to a particle having the size ranging from about 1 µm to about 999 µm.

In the present disclosure the term “about” can allow for a degree of variability in a value or range, for example, within 20%, within 10%, within 5%, or within 1% of a stated value or of a stated limit of a range.

In the present disclosure the term “substantially” can allow for a degree of variability in a value or range, for example, within 80%, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington’s Pharmaceutical Sciences, 18th ed., Mack Publishing Company, Easton, Pa., 1990, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger’s Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 µg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

In some illustrative embodiments, this present disclosure relates to a process for manufacturing a microparticle composite of a polymyxin and a Cystic Fibrosis (CF) transmembrane conductance regulator (CFTR) activator or a CFTR potentiator comprising the steps
a. preparing a suspension of nanoparticles (NPs) of a CFTR activator or a CFTR potentiator;
b. mixing the suspension of NPs with a solution of a polymyxin to afford a mixture suspension;
c. adding the mixture suspension of NPs and the polymyxin to a solution of leucine to afford a solution/suspension of NPs, the polymyxin, and leucine; and
d. spraying freeze dry, spraying dry, or freeze drying the solution/suspension of NPs, the polymyxin, and leucine to afford said micro-particle composite.

In some illustrative embodiments, this present disclosure relates to a process for manufacturing a microparticle composite of a polymyxin and a Cystic Fibrosis (CF) transmembrane conductance regulator (CFTR) activator or a CFTR potentiator as disclosed herein, wherein said microparticle composite is a dry powder inhaler (DPI) for inhalation.

In some illustrative embodiments, this present disclosure relates to a process for manufacturing a microparticle composite of a polymyxin and a Cystic Fibrosis (CF) transmembrane conductance regulator (CFTR) activator or a CFTR potentiator as disclosed herein, wherein said microparticle composite provides a way of co-delivery of a polymyxin and a CFTR activator or a CFTR potentiator.

In some illustrative embodiments, this present disclosure relates to a process for manufacturing a microparticle composite of a polymyxin and a Cystic Fibrosis (CF) transmembrane conductance regulator (CFTR) activator or a CFTR potentiator as disclosed herein, wherein said microparticle composite provides a way of co-delivery of a polymyxin and a CFTR activator or a CFTR potentiator for treatment of a lung infection or cystic fibrosis (CF).

In some illustrative embodiments, this present disclosure relates to a process for manufacturing a microparticle composite of a polymyxin and a Cystic Fibrosis (CF) transmembrane conductance regulator (CFTR) activator or a CFTR potentiator as disclosed herein, wherein said polymyxin is polymyxin B, colistin, or a pharmaceutically acceptable salt thereof.

In some illustrative embodiments, this present disclosure relates to a process for manufacturing a microparticle composite of a polymyxin and a Cystic Fibrosis (CF) transmembrane conductance regulator (CFTR) activator or a CFTR potentiator as disclosed herein, wherein said CFTR activator or CFTR potentiator is ivacaftor, elexacaftor, tezacaftor, lumacaftor, or a pharmaceutically acceptable salt thereof.

In some illustrative embodiments, this present disclosure relates to a process for manufacturing a microparticle composite of a polymyxin and a Cystic Fibrosis (CF) transmembrane conductance regulator (CFTR) activator or a CFTR potentiator as disclosed herein, wherein said microparticle composite is a dry powder inhaler (DPI) for inhalation.

In some illustrative embodiments, this present disclosure relates to a process for manufacturing a microparticle composite of a polymyxin and a Cystic Fibrosis (CF) transmembrane conductance regulator (CFTR) activator or a CFTR potentiator as disclosed herein, wherein said leucine is L-leucine, D-leucine, an oligomer of L-leucine or D-leucine, or a pharmaceutically acceptable salt thereof.

In some illustrative embodiments, this present disclosure relates to a microparticle composite manufactured according to the process disclosed herein.

In some illustrative embodiments, this present disclosure relates to a pharmaceutical product manufactured according to the process as disclosed herein.

In some illustrative embodiments, this present disclosure relates to a pharmaceutical composition comprising the microparticle composite manufactured according to the process as disclosed herein, together with one or more pharmaceutically acceptable excipients.

In some illustrative embodiments, this present disclosure relates to a dry powder inhaler product manufactured according to the process as disclosed herein.

In some illustrative embodiments, this present disclosure relates to a method for treating a patient of cystic fibrosis (CF) or lung infection comprising the step of administrating a therapeutic effective amount of a product manufactured according to the process as disclosed herein, to the patient in need of relief from said CF or lung infection.

In some illustrative embodiments, this present disclosure relates to a microparticle composite or a dry powder inhaler comprising a polymyxin and a CFTR activator or a CFTR potentiator manufactured according to the steps of
a. preparing a suspension of nanoparticles (NPs) of said CFTR activator or said CFTR potentiator;
b. mixing the suspension of NPs with a solution of said polymyxin to afford a mixture suspension;
c. adding the mixture suspension of NPs and said polymyxin to a solution of leucine to afford a solution/suspension of NPs, said polymyxin, and leucine;
d. spraying freeze dry, spraying dry or freeze drying said solution/suspension to afford said micro-particle composite.

In some other illustrative embodiments, this present disclosure relates to a microparticle composite or a dry powder inhaler comprising a polymyxin and a CFTR activator or a CFTR potentiator manufactured according to the steps as disclosed herein, wherein said microparticle composite is a dry powder inhaler (DPI) for inhalation.

In some other illustrative embodiments, this present disclosure relates to a microparticle composite or a dry powder inhaler comprising a polymyxin and a CFTR activator or a CFTR potentiator manufactured according to the steps as disclosed herein, wherein said microparticle composite provides a way of co-delivery of a polymyxin and a CFTR activator or a CFTR potentiator.

In some other illustrative embodiments, this present disclosure relates to a microparticle composite or a dry powder inhaler comprising a polymyxin and a CFTR activator or a CFTR potentiator manufactured according to the steps as disclosed herein, wherein said microparticle composite provides a way of co-delivery of a polymyxin and a CFTR activator or a CFTR potentiator for treatment of a lung infection or cystic fibrosis (CF).

In some other illustrative embodiments, this present disclosure relates to a microparticle composite or a dry powder inhaler comprising a polymyxin and a CFTR activator or a CFTR potentiator manufactured according to the steps as disclosed herein, wherein said polymyxin is polymyxin B, colistin, or a pharmaceutically acceptable salt thereof.

In some other illustrative embodiments, this present disclosure relates to a microparticle composite or a dry powder inhaler comprising a polymyxin and a CFTR activator or a CFTR potentiator manufactured according to the steps as disclosed herein, wherein said CFTR activator or CFTR potentiator is ivacaftor, elexacaftor, tezacaftor, lumacaftor, or a pharmaceutically acceptable salt thereof.

In some other illustrative embodiments, this present disclosure relates to a microparticle composite or a dry powder inhaler comprising a polymyxin and a CFTR activator or a CFTR potentiator manufactured according to the steps as disclosed herein, wherein said leucine is L-leucine, D-leucine, an oligomer of L-leucine or D-leucine, or a pharmaceutically acceptable salt thereof.

In some other illustrative embodiments, this present disclosure relates to a pharmaceutical composition comprising the microparticle composite or dry powder inhaler as disclosed herein, together with one or more pharmaceutically acceptable excipients.

In some other illustrative embodiments, this present disclosure relates to a method for treating a subject with a lung infection or CF comprising the step of administrating a therapeutically effective amount of a pharmaceutical composition as disclosed herein, to the subject in need of relief from said CF or lung infection.

In some other illustrative embodiments, this present disclosure relates to a pharmaceutical composition comprising the microparticle composite or dry powder inhaler as disclosed herein, together with one or more pharmaceutically acceptable excipients, for use as a medicament for treatment of cystic fibrosis (CF) or a lung infection.

The following detailed examples are provided for better understanding the scopes and utilities of the present disclosure. They should not be considered in any way as to limit the scopes of the present disclosure.

Pulmonary drug delivery system (PDDS) can directly deliver drugs to the lung to increase the local drug concentration and limit systemic adverse effects (de Boer et al., 2012; Grasmeijer et al., 2014; Smyth and Hickey, 2005). Among PDDS technologies, dry powder inhalers (DPIs) exhibit advantageous portability and stability (Elsayed and Shalash, 2018) (Lin et al., 2015). The co-delivery DPIs containing Iva and Col is a promising approach for the treatment of CF and its complications of lung infections. However, the solubility properties of Iva and Col vary widely; the former is hydrophobic and the latter is hydrophilic. It is necessary to improve the aqueous solubility of Iva so as to enhance its dissolution and activity in the lungs.

In recent years, nanoparticles have shown potential as drug carrier systems to improve drug physicochemical properties and present several advantages such as drug protection, controlled drug release and enhanced binding capacity of various drugs (Casa et al., 2015; Fonseca et al., 2017). Bovine serum albumin (BSA) is widely used as a nanocarrier due to its low cost, safety and non-immunogenicity (Casa et al., 2018; Elzoghby et al., 2012). In addition, due to flexibility of BSA structure, together with the different charged molecules, BSA could bind many compounds with different structures and solubility characteristics (Fang et al., 2011). Moreover, the unique ligand-binding properties of BSA could enhance the solubility of the conjugated hydrophobic drugs and improve the bioactivity of drug molecules in a biological environment (Singh et al., 2017).

Previous studies have shown that combining Colistin (Col), a cationic polypeptide antibiotic, with Ivacaftor (Iva), a cystic fibrosis (CF) drug, could achieve synergistic antibacterial effects against Pseudomonas aeruginosa. The purpose of this study was to develop dry powder inhaler (DPI) formulations for co-delivery of Col and Iva, aiming to treat CF and lung infection simultaneously. In order to improve solubility and dissolution for the water insoluble Iva, it was encapsulated into bovine serum albumin (BSA) nanoparticles (Iva-BSA-NPs). Inhalable composite of Iva-BSA-NPs using water-soluble Col as the matrix material and L-leucine as aerosol enhancer were produced by the spray-freeze-drying technique. The optimal formulation showed irregular-shaped morphology with the fine particle fraction (FPF) value of 73.75 ± 5.23% for Col and 80.91 ± 4.10% for Iva. Iva-BSA-NPs were amorphous and remained in the amorphous state after spray-freeze-drying as examined by powder X-ray diffraction. In vitro dissolution profiles of the DPI formulation indicated that Col and Iva were almost completely released within 3 hours, which were substantially faster regarding Iva release than the jet milled physical mixture of two jet-milled drugs. In summary, this study developed a novel inhalable nano-composite microparticle using a synergistic water-soluble drug as the matrix material, which achieved reduced use of excipients for high-dose medications, improved dissolution rate for the water-insoluble drug and superior aerosol performance.

### 2. Materials and Methods

### 2.1. Materials

Ivacaftor (Iva) and colistin (Col) were purchased from AOKChem (Shanghai, China) and Betapharma Co. Ltd. (Jiangsu, China), respectively. Bovine serum albumin (BSA, purity > 98%, Mw 66.5 kDa), glutaraldehyde and L-leucine were supplied by Sigma-Aldrich Inc. (St. Louis, USA). 3-(4, 5-dimethylthiazol-2-yl)-2, 5-diphenyltetrazolium bromide (MTT) and acetonitrile (HPLC grade) were obtained from Thermo Fisher Scientific Inc. (Massachusetts, USA). The fetal bovine serum and Dulbecco’s Modified Eagle Medium (DMEM) were purchased from Gibco Life Technologies Corporation (Eugene, USA). All other reagents and chemicals were analytical grade.

### 2.2. Preparation of Iva-BSA-NPs

Iva-BSA-NPs were prepared by the de-solvation method with some modifications (Bhushan et al., 2015; Fonseca et al., 2017; Kayani et al., 2018). Initially, BSA and Iva with the mass ratio of 20:2, 25:2 or 30:2 were dissolved in ultra-pure water and dimethyl sulfoxide (DMSO), respectively. The volume ratio of aqueous phase and organic phase was set as 3:1. Then the nanoparticles were obtained by a continuous dropwise addition of Iva solution into BSA solution with a constant rate of 1.0 mL/min under an ultrasonic dispersion (Ultrasonic Bath 5.7 L, Thermo Fisher scientific Inc.). After the de-solvation process, glutaraldehyde solution (8.0%, v/v) was added into nano-suspension with the volume ratio of 1:100 and magnetic stirring (400 rpm) for 24 hours at ambient temperature to full crosslinking of the amino groups of BSA. Finally, the resulting Iva-BSA-NPs were obtained by centrifugation (16,000 g for 20 min), being washed three times with ultra-pure water to eliminate the free BSA, Iva and excess glutaraldehyde.

### 2.3. Preparation of DPI Formulations by Spray Freeze Drying for Co-Delivery Col and Iva-BSA-NPs

The co-delivery DPI formulations were prepared by spray freeze drying. L-leucine was used as the matrix and aerosol enhancer excipient in all formulations. The preparation process was slightly modified from the previous studies (Ali and Lamprecht, 2014; Liao et al., 2019). Col solution (4.0 mg/mL) was mixed with the equal volume of Iva-BSA-NPs suspension (4.0 mg/mL) to obtain the suspensions. Then these suspensions were added to the L-Leucine solution to make the total solid contents respective as 1.0%, 2.0%, 3.0%, 5.0%, 8.0% and 10.0% w/w, which were named as F1 to F6. Afterwards, the suspension was pumped through an ultrasonic atomizer nozzle (9230 Flawil, Switzerland) into liquid nitrogen with the power of 3.5 watt at a controlled feed rate of 2.0 mL/min. Subsequently, the atomized droplets were frozen, collected and transferred to the Labconco freeze dryer under vacuum (chamber pressure below 0.5 mbar) at -25° C. for 48 hours to achieve the sublimation of solvents. Finally, the microparticles were collected and sealed in the desiccator with silica beads for further studies.

### 2.4. HPLC Analysis

Iva and Col were analyzed by high performance liquid chromatography (HPLC, 1260, Agilent, Germany) as described previously with some modifications (Akram and Umamahesh, 2017; Wang et al., 2018). An Eclipse plus C18 column (4.6 ×150 mm, 5 µm, Agilent, Santa Clara, USA) was employed for the simultaneous determination of Iva and Col. The mobile phase for Col was a mixture of 30 mM sodium sulfate solution (pH adjusted to 2.5 with H₃PO₄, 76% v/v) and acetonitrile (24% v/v), while for Iva was acetonitrile/water/methanol (3:1:0.17) mixture with pH 3.0 (pH adjusted with triethylamine). Samples were eluted at a flow rate of 1.0 mL/min, and the wavelength was set at 215 nm and 254 nm for Col and Iva, respectively. The injection volume was 20 µL and the total run time was 30 min. The linearity was found in the concentration range of 0.5-100.0 µg/mL and 0.5-200.0 µg/mL for Col and Iva with the correlation coefficient (R²) of 0.995 and 0.999, respectively.

### 2.5. Characterization of Iva-BSA-NPs

### 2.5.1. Particle Size and Zeta Potential (ZP)

The prepared Iva-BSA-NPs were analyzed regarding particle size, polydispersity index (PDI) and ZP by Malvern Nano Series (ZS90, Malvern Instruments Ltd., Malvern, UK). Each parameter was measured in triplicate.

### 2.5.2. Encapsulation Efficiency (EE%) of Nanoparticles

During the preparation of Iva-BSA-NPs, the free Iva in the supernatant after centrifugation was collected and analyzed by HPLC. Then the EE% was calculated by the follow equation.

$$\text{EE} \% = \left(1 - \frac{{\text{M}}_{\text{free} \text{Iva}}}{{\text{M}}_{\text{total} \text{Iva}}}\right) \times 100 \%$$

wherein the M_{free Iva} and M_{total Iva} represent the uncross-linked and the originally added drugs, respectively.

### 2.5.3. Morphology of Nanoparticles

In order to examine the morphology of the optimal nanoparticles, the freeze dryer (Free zone 4.5, Labconco, Kansas City, USA) was used to lyophilize nanoparticles after they were washed and collected. Samples were fixed on the double-sided tape and gold-coated at 40 mA for 60 s using a sputter coater (208 HR, Cressington Scientific Instruments, Watford, UK). Images were captured by a field emission scanning electron microscope (NOVA nano SEM, FEI Company, USA) at an acceleration voltage of 10.0 kV.

### 2.5.4. Crystallinity of Nanoparticles

Crystalline state of the optimal Iva-BSA-NPs and the physical mixture (PM) of BSA and Iva were characterized by the powder X-ray diffraction (PXRD, SmartLab™ diffractometer, Rigaku Americas, Austin, USA) with Cu-Kα radiation source, operating at 40 kV and 44 mA . The scanning 2θ range was set as 5 to 60° with a scanning rate of 5°/min (Mangal et al., 2018a).

### 2.6. Characterization of DPI Formulations

### 2.6.1. Particle Size, Tapped Density and True Density

The particle size of F1 to F6 was measured by a Malvern MasterSizer 3000 (Malvern Instruments, Malvern, UK) using a dry dispersion unit with air pressure of 2.0 bar, refractive index of 1.65 and feed rate vibration of 50%. The Sₚₐₙ values were calculated by Eq. 2 based on D₉₀, D₁₀ and D₅₀, which represent the volume diameter of particles at 90%, 10% and 50%, respectively. Samples were analyzed in triplicate.

$${\text{S}}_{\text{pan}} = \frac{{\text{D}}_{90} - {\text{D}}_{10}}{{\text{D}}_{50}}$$

Wherein the true density of the co-delivery microparticles were determined by a Micromeritic Pycnometer (AccuPyc 1340, Micromeritics, USA). Each measurement was repeated five times.

### 2.6.2. Crystallinity of DPI Formulations

Crystalline state of co-delivery microparticles and the PM of BSA, Iva, Col and L-leucine were characterized by PXRD with the similar method as to detect the optimal nanoparticles.

### 2.6.3. Morphology of DPI Formulations

Morphology of the co-delivery microparticles (F1~F6) were also observed by SEM with the similar method mentioned above for nanoparticles. The SEM images were captured using the inbuilt software and appropriate magnification was selected for each image.

### 2.6.4. In Vitro Aerosolization

In vitro aerosolization properties of DPI formulations were assessed using a Multi-Stage Liquid Impinger (MSLI) (Copley Scientific Limited, Nottingham, UK). Each sample (approximately 10 ± 2 mg) was loaded into #3 hydroxypropyl methylcellulose capsules (Qualicaps, Whitsett, USA). Five capsules were dispersed through an low-resistant RS01 DPI device (Plastiape S.p.A., Osnago, Italy) using a standard dispersion procedure of 2.4 s at 100 L/min flow rate to generate approximately 4 kPa pressure drop cross the device (Mangal et al., 2018b). The cutoff diameters for Stages 1 to 4 of the MSLI were 10.4, 4.9, 2.4, and 1.2 µm, respectively (Mangal et al., 2018b). Drugs retained in each part of dispersion equipment were washed and dissolved by the Iva mobile phase. Emitted dose (ED) and fine particle fraction (FPF) was respectively calculated by Eq. 3 and Eq. 4. Each sample was evaluated in triplicate.

$$\text{ED} \% = \frac{\text{Throat} \text{+} {\text{S}}_{1} + {\text{S}}_{2} + {\text{S}}_{3} + {\text{S}}_{4} + \text{Filter}}{\text{Capsule} \text{+} \text{Device} \text{+} \text{Throat} + {\text{S}}_{1} + {\text{S}}_{2} + {\text{S}}_{3} + {\text{S}}_{4} + \text{Filter}} \times 100 \%$$

$$\text{FPF} \% = \frac{{\text{S}}_{3} + {\text{S}}_{4} + \text{Filter}}{\text{Throat} + {\text{S}}_{1} + {\text{S}}_{2} + {\text{S}}_{3} + {\text{S}}_{4} + \text{Filter}} \times 100 \%$$

### 2.7. Dissolution Tests

Dissolution experiments were performed at 37.0 ± 1.0° C. using a Franz cell (V6B, PermeGear Inc., USA) with the preset stirring speed of 600 rpm. Samples were fired into the next generation impactor (NGI, Copley Scientific Ltd., UK) with the flow rate of 100 L/min for 2.4 s to collect the aerosolized particles in S4 plate (the cut-off mean aerodynamic diameter is 1.31 µm) with a membrane (Whatman, Buckinghamshire, UK). Twenty milliliter of phosphate buffered saline (PBS pH 7.4) with 0.5% SDS was used as the in vitro dissolution medium. The membrane containing the aerosolized powder was placed on the top of the Franz cell and fixed by a holder and a clamp, being in contact with the dissolution media (May et al., 2012; Wang et al., 2016). At each time points of 5, 10, 20, 30, 60, 120, 180 and 360 min, 100 µL of dissolution medium was withdrawn and equal volume of fresh media was added to maintain a constant dissolution volume. After the last time point, the membrane was rinsed by the dissolution media in the cell reservoirs as the total accumulated dose. The contents of Col and Iva were determined by HPLC, and each test was performed in triplicate.

### 2.8. Statistical Analysis

The data were reported as mean ± standard deviation (SD). Statistical analysis was conducted by SPSS 19.0 software (IBM Corporation, USA) using one-way analysis of variance (ANOVA). The difference between variants was considered significant if P <0.05.

### 3. Results and Discussion

### 3.1. Characterization of Iva-BSA-NPs

### 3.1.1. Particle Size, ZP and EE%

The main characteristics of Iva-BSA-NPs were presented in Table 1. The mean particle size of Iva-BSA-NPs were all below 250 nm with a relatively narrow size distribution, which characterized by PDI values below 0.3. ZP was negative due to the charged groups of BSA and the negative charge was increased with an increase in the mass ratio of BSA. ZP is important to reduce the tendency to aggregate for the nano-suspension and therefore to maintain the physical stability (Nosrati et al., 2018). Moreover, the negative surface charge of nanoparticles may enhance interactions with the positively charged Col through electrostatic charge in the preparation of composite microparticles.

The EE% of Iva-BSA-NPs was higher than 70% due to the high binding capacity of two main binding sites (sites I and II) of BSA with small molecule drugs (Kayani et al., 2018). The BSA to Iva ratio of 25:2 was selected as the optimal formulation for subsequent studies considering its size, PDI, EE and drug loading.

Table 1

Physical-chemical characteristics of Iva-BSA-NPs (n=3)

| BSA: Iva (w/w) | Particle size (nm) | PDI | ZP (mV) | EE% |
|---|---|---|---|---|
| 20:2 | 234.5±6.5 | 0.22±0.09 | -20.3±2.8 | 73.1±3.6 |
| 25:2 | 173.2±4.1 | 0.13±0.04 | -27.9±3.0 | 78.7±2.6 |
| 30:2 | 171.5±4.3 | 0.14±0.03 | -30.5±2.9 | 79.4±2.5 |

### 3.1.2. Morphology and Crystallinity of Nanoparticles

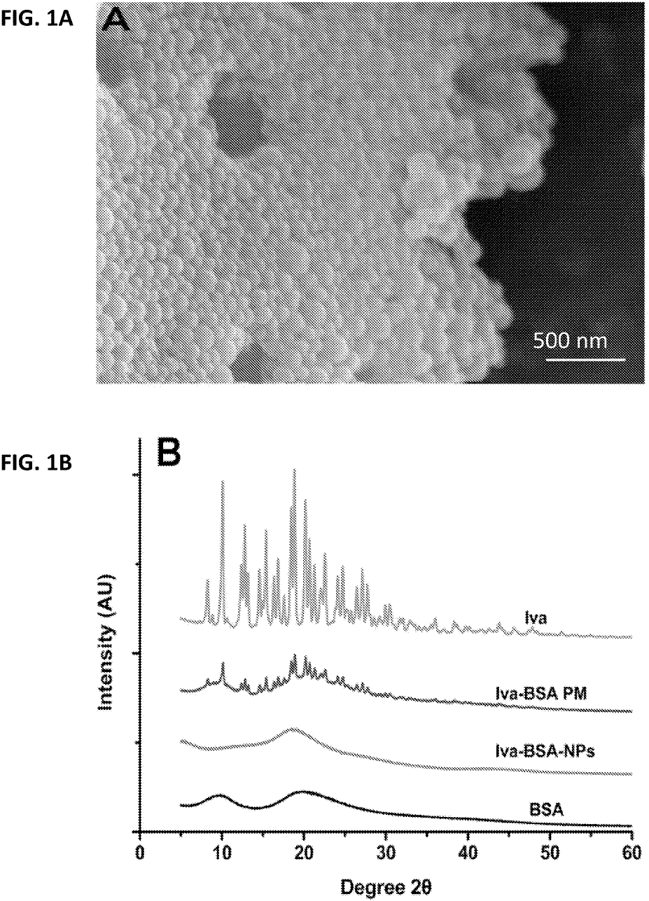

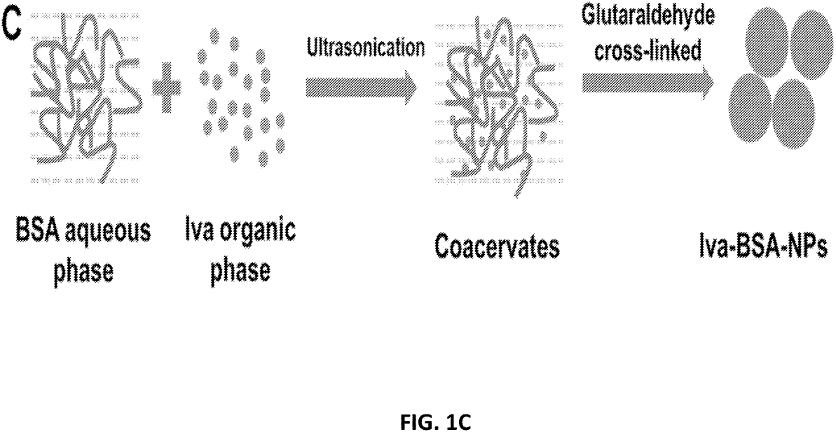

Representative SEM image of Iva-BSA-NPs (BSA: Iva=25:2) was shown in FIG. 1A, which presents a spherical shape with the particle size below 200 nm and relatively uniform particle size distribution. PXRD diffractogram of the raw Iva and PM of BSA and Iva showed sharp peaks indicating they were crystalline (FIG. 1B). In contrast, Iva-BSA-NPs did not exhibit any crystalline peaks, suggesting Iva was amorphous when prepared to nanoparticles.

The formation mechanism of Iva-BSA-NPs was showed in FIG. 1C. In the de-solvation process, with the addition of organic solvent into the BSA solution, BSA was phase separated due to its diminished solubility (Langer et al., 2003). Subsequently, based on the binding of protein with small drug molecules via Van der Waals interactions and hydrogen bonds, the coacervates were formed (Bhushan et al., 2015; Fonseca et al., 2017; Singh et al., 2017). After that, the coacervates were further hardened, attributed to the condensation reactions between aldehyde group of glutaraldehyde and amino moieties of lysine residues or guanidino moieties of arginine residues (Gawde et al., 2018; Merodio et al., 2001). More importantly, this process converted the crystal form of Iva to amorphous glassy state, which could enhance water solubility and dissolution of poorly water soluble Iva.

### 3.2. Characterization of DPI Formulations

### 3.2.1. Particle Size, Tapped Density and True Density

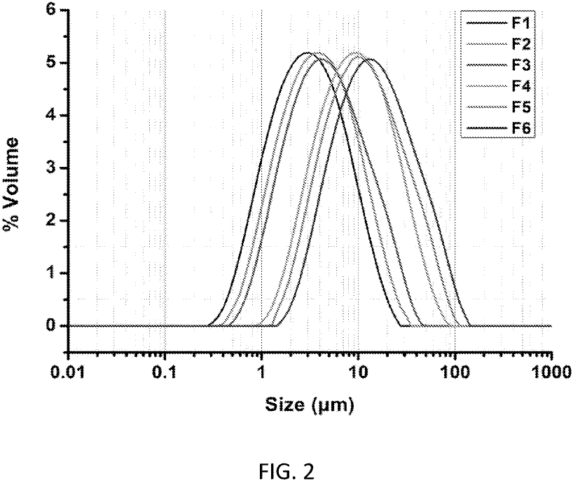

The particle sizes of powder formulations with different solid contents were shown in FIG. 2. With the increase in solid content, the mean particle diameter was also increased. All formulations exhibited Gaussian distribution with the Sₚₐₙ value less than 2.0, which indicated a satisfactory uniformity of particle size.

Based on the D_{[4,3]} and the measurement of tapped density, the theoretical (or calculated) particle aerodynamic diameter (Dₐₑ) increased with an increase in the solid content (Table 2). Similarly, the true density also increased from 0.29 ± 0.01 to 0.77 ± 0.02 g/cm³.

Table 2

physical-chemical characteristics and in vitro aerosolization properties of co-delivery DPI formulations with different solid contents (mean ± SD).

| Solid content (%) | Ture density (g/cm³)^{a} | ED (Col)^{b} | FPF (Col)^{b} | ED (Iva)^{b} | FPF (Iva)^{b} |
|---|---|---|---|---|---|
| F1 1.0 | 0.29±0.01 | 95.78±2.04 | 67.83±3.70 | 97.52±1.07 | 75.61±1.02 |
| F2 2.0 | 0.31±0.01 | 97.44±1.46 | 73.75±5.23 | 96.42±1.99 | 80.91±4.10 |
| F3 3.0 | 0.32±0.02 | 95.29±1.92 | 56.50±1.98 | 96.20±2.86 | 63.23±2.15 |
| F4 5.0 | 0.43±0.01 | 96.45±3.35 | 46.35±6.98 | 96.36±3.23 | 38.75±8.81 |
| F5 8.0 | 0.56±0.01 | 98.97±1.61 | 27.98±9.75 | 94.71±3.54 | 18.21±6.61 |
| F6 10.0 | 0.77±0.02 | 94.89±2.33 | 25.17±2.05 | 91.89±1.31 | 17.44±1.59 |

^{a}n=5; ^{b}n=3

### 3.2.2. Crystallinity of DPI Powders

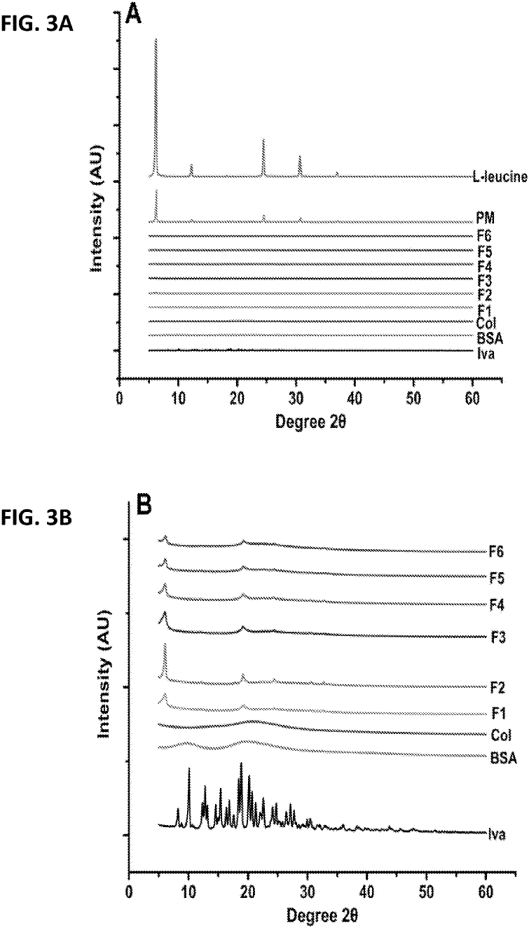

The PXRD results for powder formulations (F1~F6), raw drugs (Iva and Col), BSA, L-leucine and PM are showed in FIG. 3A. Because the sharp crystalline peaks of L-leucine masked the peaks of other materials, L-leucine and PM results were excluded to obtain FIG. 3B. It is clear that sharp peaks shown in pure Iva are not observed in F1 to F6, indicating that Iva was amorphous in the DPI formulations. These results were consistent with the PXRD data for nanoparticles.

### 3.2.3. Morphology of DPI Formulations

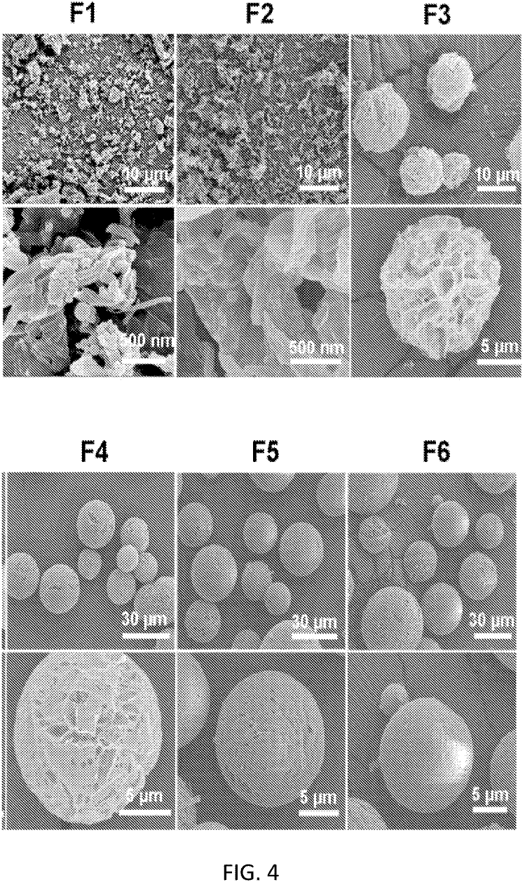

FIG. 4 showed SEM micrographs of DPI formulations (F1~F6) at two different magnifications. Iva-BSA-NPs were visible in the microparticles at least for F1 and F2.

The effect of solid content on the morphology of powder is apparent. As the solid content increased from 1.0% to 10.0%, the morphology of particles changed from a loose irregular shape to spherical. This is because at low solid contents, particles are very porous which collapse during lyophilization under vacuum.

### 3.2.4. In Vitro Aerosolization Properties of DPI Formulations

Table 2 displays the in vitro aerosolization properties of DPI formulations. The ED values of all formulations were higher than 90% and there was no significant difference between the formulations with different solid contents (p>0.05). An increase in solid contents led to higher FPF values for both Col and Iva, except for F1. It is interesting that particles of F1 and F2 collapsed during freeze drying under vacuum. However, the irregular shape of collapsed particles did not compromise the FPF. The microparticles with lower solid contents have a lower density, which leads to smaller Dₐₑ values and better aerosol performance (D′ Addio et al., 2012; Frijlink and De Boer, 2004; Rahimpour et al., 2014). F2 showed the highest FPF value of 73.75±5.23% for Col and 80.91±4.10% for Iva, which was selected as the optimal formulation for the dissolution study.

### 3.3. Dissolution Tests

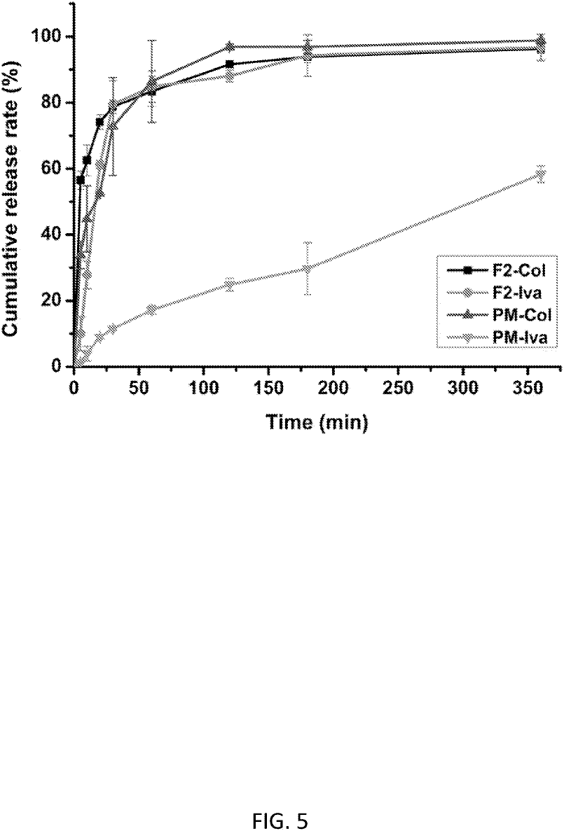

The formulation with the solid content of 2.0% (F2) was selected for dissolution test under the sink condition. The jet-milled PM of F2 was used as the control group. Dissolution profiles of F2 and the jet-milled PM under sink conditions were shown in FIG. 5. For the jet-milled PM, only 29.71 ± 7.86% of Iva was dissolved within 180 min and less than 60% after 6 h. This is attributed to the high hydrophobicity and low aqueous solubility of Iva molecules (Lin et al., 2017). In contrast, about 94% of Iva was dissolved in 3 h for F2, and the dissolution rate was similar to the water-soluble Col. Such substantially enhanced dissolution is attributed to the amorphous form and nano-meter particle size of Iva in the Iva-BSA-NPs. Water-soluble material such as colistin also provides a fast-dissolving matrix to avoid the aggregation and facilitate the dispersion of nanoparticles in the dissolution medium.

4. Conclusions. Pulmonary delivery of the hydrophilic drug of Col and the hydrophobic drug of Iva by dry powder inhalers directly to the lungs could be effective in treating cystic fibrosis and its complications such as resistant lung infections. In this study, Iva-BSA-NPs were generated by de-solvation method to convert the crystalline form of Iva to amorphous nanoparticles with a purpose to enhance its dissolution. Novel inhalable nano-composite microparticle formulations were developed by dispersing Iva-BSA-NPs in a synergistic water-soluble colistin as the matrix material to achieve co-delivery. It was demonstrated that the solid content significantly affected the physico-chemical properties of DPI formulations, including morphology, particle size, true density, and especially the aerosolization. The optimal composite DPI formulation showed an irregular-shaped morphology and excellent aerosolization performance with the FPF value of 73.75±5.23% for Col and 80.91±4.10 for Iva. More importantly, the formulation achieved up to 94% dissolution for Iva within 3 h, which has a comparable dissolution rate to the water-soluble Col in the in vitro dissolution test. Such synchronized dissolution behavior of two synergistic drugs could potentially be converted into superior bioactivity.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES CITED

1. Akram, N.M., Umamahesh, M., 2017. A New Validated RP-HPLC Method for the Determination of Lumacaftor and Ivacaftor in its Bulk and Pharmaceutical Dosage Forms. Orient J Chem 33, 1492-1501.
2. Ali, M.E., Lamprecht, A., 2014. Spray freeze drying for dry powder inhalation of nanoparticles. Eur J Pharm Biopharm 87, 510-517.
3. Bhushan, B., Dubey, P., Kumar, S.U., Sachdev, A., Matai, I., Gopinath, P., 2015. Bionanotherapeutics: niclosamide encapsulated albumin nanoparticles as a novel drug delivery system for cancer therapy. Rsc Adv 5, 12078-12086.
4. Casa, D.M., Karam, T.K., Alves, A.D.S., Zgoda, A.A., Khalil, N.M., Mainardes, R.M., 2015. Bovine Serum Albumin Nanoparticles Containing Amphotericin B: Characterization, Cytotoxicity and In Vitro Antifungal Evaluation. J Nanosci Nanotechno 15, 10183-10188.
5. Casa, D.M., Scariot, D.B., Khalil, N.M., Nakamura, C.V., Mainardes, R.M., 2018. Bovine serum albumin nanoparticles containing amphotericin B were effective in treating murine cutaneous leishmaniasis and reduced the drug toxicity. Exp Parasitol 192, 12-18.
6. d’Angelo, I., Casciaro, B., Miro, A., Ungaro, F., 2015. Overcoming barriers in Pseudomonas aeruginosa lung infections: Engineered nanoparticles for local delivery of a cationic antimicrobial peptide. Colloid Surface B 135, 717-725.
7. D’Addio, S.M., Chan, J.G.Y., 2012. Constant size, variable density aerosol particles by ultrasonic spray freeze drying. 427, 185-191.
8. de Boer, A.H., Chan, H.K., Price, R., 2012. A critical view on lactose-based drug formulation and device studies for dry powder inhalation: Which are relevant and what interactions to expect? Advanced Drug Delivery Reviews 64, 257-274.
9. E. H. Gnagne, J.P., C. Gaiani, J. Scher, G. N. Amani, 2017. Characterisation of flow properties of foutou and foufou flours, staple foods in west africa, using the FT4 powder rheometer. Food Measure 11, 1128-1136.
10. Elsayed, M.M.A., Shalash, A.O., 2018. Modeling the performance of carrier-based dry powder inhalation formulations: Where are we, and how to get there? J Control Release 279, 251-261.
11. Elzoghby, A.O., Samy, W.M., Elgindy, N.A., 2012. Albumin-based nanoparticles as potential controlled release drug delivery systems. J Control Release 157, 168-182.
12. Fang, R., Hao, R.F., Wu, X., Li, Q., Leng, X.J., Jing, H., 2011. Bovine Serum Albumin Nanoparticle Promotes the Stability of Quercetin in Simulated Intestinal Fluid. J Agr Food Chem 59, 6292-6298.
13. Fonseca, D.P., Khalil, N.M., Mainardes, R.M., 2017. Bovine serum albumin-based nanoparticles containing resveratrol: Characterization and antioxidant activity. J Drug Deliv Sci Tec 39, 147-155.
14. Freeman, R., 2007. Measuring the flow properties of consolidated, conditioned and aerated powders - A comparative study using a powder rheometer and a rotational shear cell. Powder Technol 174, 25-33.
15. Frijlink, H.W., De Boer, A.H., 2004. Dry powder inhalers for pulmonary drug delivery. Expert Opin Drug Deliv 1, 67-86.
16. Garbuzenko, O.B., Kbah, N., Kuzmov, A., Pogrebnyak, N., Pozharov, V., Minko, T., 2019. Inhalation treatment of cystic fibrosis with lumacaftor and ivacaftor co-delivered by nanostructured lipid carriers. J Control Release 296, 225-231.
17. Garonzik, S.M., Li, J., Thamlikitkul, V., Silveira, F.P., Forrest, A., Nation, R.L., 2011. Population Pharmacokinetics of Colistin Methanesulfonate and Formed Colistin in Critically Ill Patients from a Multicenter Study Provide Dosing Suggestions for Various Categories of Patients. Antimicrob Agents Ch 55, 3284-3294.
18. Gawde, K.A., Sau, S., Tatiparti, K., Kashaw, S.K., Mehrmohammadi, M., Azmi, A.S., Iyer, A.K., 2018. Paclitaxel and di-fluorinated curcumin loaded in albumin nanoparticles for targeted synergistic combination therapy of ovarian and cervical cancers. Colloid Surface B 167, 8-19.
19. Grasmeijer, F., Lexmond, A.J., van den Noort, M., Hagedoorn, P., Hickey, A.J., Frijlink, H.W., de Boer, A.H., 2014. New mechanisms to explain the effects of added lactose fines on the dispersion performance of adhesive mixtures for inhalation. PloS one 9, e87825.
20. Hamilton, C.M., Hung, M., Chen, G., Qureshi, Z., Thompson, J.R., Sun, B.Y., Bear, C.E., Young, R.N., 2018. Synthesis and characterization of a photoaffinity labelling probe based on the structure of the cystic fibrosis drug ivacaftor. Tetrahedron 74, 5528-5538.
21. Hubert, D., Chiron, R., Camara, B., Grenet, D., Prevotat, A., Bassinet, L., Dominique, S., Rault, G., Burgel, P.R., 2017. Real-life initiation of lumacaftor/ivacaftor combination in adults with cystic fibrosis homozygous for the Phe508del CFTR mutation and severe lung disease. J Cyst Fibros 16, 388-391.
22. Katherine Fesen, P.S., Nathalie Fuentes, Marvin Nicoleau, Lidys Rivera, Diane Kitch, Gavin R. Graff, Roopa Siddaiah, 2019. The role of microRNAs in chronic pseudomonas lung infection in Cystic fibrosis. Respiratory Medicine 151, 133-138.
23. Kayani, Z., Firuzi, O., Bordbar, A.K., 2018. Doughnut-shaped bovine serum albumin nanoparticles loaded with doxorubicin for overcoming multidrug-resistant in cancer cells. Int J Biol Macromol 107, 1835-1843.
24. Langer, K., Balthasar, S., Vogel, V., Dinauer, N., von Briesen, H., Schubert, D., 2003. Optimization of the preparation process for human serum albumin (HSA) nanoparticles. Int J Pharmaceut 257, 169-180.
25. Liao, Q.Y., Yip, L., Cho, M.Y.T., Chow, S.F., Chan, H.K., Lam, J.K.W., 2019. Porous and highly dispersible voriconazole dry powders produced by spray freeze drying for pulmonary delivery with efficient lung deposition. Int J Pharmaceut 560, 144-154.
26. Lin, L., Quan, G.L., Peng, T.T., Huang, Z.W., Singh, V., Lu, M., Wu, C.B., 2017. Development of fine solid-crystal suspension with enhanced solubility, stability, and aerosolization performance for dry powder inhalation. Int J Pharmaceut 533, 84-92.
27. Lin, Y.W., Wong, J., Qu, L., Chan, H.K., Zhou, Q.T., 2015. Powder production and particle engineering for dry powder inhaler formulations. Current pharmaceutical design 21, 3902-3916.
28. Liu, Y.H., Kuo, S.C., Yao, B.Y., Fang, Z.S., Hu, C.M.J., 2018. Colistin nanoparticle assembly by coacervate complexation with polyanionic peptides for treating drug-resistant gram-negative bacteria. Acta Biomater 82, 133-142.
29. Lu, X.Y., Chen, L., Wu, C.Y., Freeman, T., 2017. The Effects of Relative Humidity on the Flowability and Dispersion Performance of Lactose Mixtures. Materials 10.
30. Mangal, S., Nie, H.C., Xu, R.K., Guo, R., Cavallaro, A., Zemlyanov, D., Zhou, Q., 2018a. Physico-Chemical Properties, Aerosolization and Dissolution of Co-Spray Dried Azithromycin Particles with L-Leucine for Inhalation. Pharm Res-Dordr 35.
31. Mangal, S., Park, H., Zeng, L., Yu, H.H., Zhou, Q.T., 2018b. Composite particle formulations of colistin and meropenem with improved in-vitro bacterial killing and aerosolization for inhalation. Int J Pharm 548, 443-453.
32. May, S., Jensen, B., Lehr, C.M., 2012. Dissolution Techniques for In Vitro Testing of Dry Powders for Inhalation. Pharm Res-Dordr 29, 2157-2166.
33. McColley, S.A., Konstan, M.W., Ramsey, B.W., Elborn, J.S., Boyle, M.P., Wainwright, C.E., Waltz, D., Vera-Llonch, M., Marigowda, G., Jiang, J.G., Rubin, J.L., 2019. Lumacaftor/Ivacaftor reduces pulmonary exacerbations in patients irrespective of initial changes in FEV1. J Cyst Fibros 18, 94-101.
34. Merodio, M., Arnedo, A., Irache, J.M., 2001. Ganciclovir-loaded albumin nanoparticles: characterization and in vitro release properties. Eur J Pharm Sci 12, 251-259.
35. Moffatt, J.H., Harper, M., Harrison, P., Hale, J.D.F., Vinogradov, E., Seemann, T., Henry, R., Crane, B., Michael, F.S., Cox, A.D., Adler, B., Nation, R.L., Li, J., Boyce, J.D., 2010. Colistin Resistance in Acinetobacter baumannii Is Mediated by Complete Loss of Lipopolysaccharide Production. Antimicrob Agents Ch 54, 4971-4977.
36. Nosrati, H., Sefidi, N., Sharafi, A., Danafar, H., Manjili, H.K., 2018. Bovine Serum Albumin (BSA) coated iron oxide magnetic nanoparticles as biocompatible carriers for curcumin-anticancer drug. Bioorg Chem 76, 501-509.
37. Porsio, B., Craparo, E.F., Cavallaro, G., 2018. Mucus and Cell-Penetrating Nanoparticles Embedded in Nano-into-Micro Formulations for Pulmonary Delivery of Ivacaftor in Patients with Cystic Fibrosis. Acs Appl Mater Inter 10, 165-181.
38. Rahimpour, Y., Kouhsoltani, M., Hamishehkar, H., 2014. Alternative carriers in dry powder inhaler formulations. Drug discovery today 19, 618-626.
39. Reznikov, L.R., Abou Alaiwa, M.H., Dohrn, C.L., Gansemer, N.D., Diekema, D.J., Stoltz, D.A., Welsh, M.J., 2014. Antibacterial properties of the CFTR potentiator ivacaftor. J Cyst Fibros 13, 515-519.
40. Schneider, E.K., Azad, M.A.K., Han, M.L., Zhou, Q., Wang, J.P., Huang, J.X., Cooper, M.A., Doi, Y., Baker, M.A., Bergen, P.J., Muller, M.T., Li, J., Velkov, T., 2016. An “Unlikely” Pair: The Antimicrobial Synergy of Polymyxin B in Combination with the Cystic Fibrosis Transmembrane Conductance Regulator Drugs KALYDECO and ORKAMBI. Acs Infect Dis 2, 478-488.
41. Singh, P., Kim, Y.J., Singh, H., Ahn, S., Castro-Aceituno, V., Yang, D.C., 2017. In situ preparation of water-soluble ginsenoside Rh2-entrapped bovine serum albumin nanoparticles: in vitro cytocompatibility studies. Int J Nanomed 12, 4073-4084.
42. Smyth, H.D., Hickey, A.J., 2005. Carriers in drug powder delivery. American Journal of Drug Delivery 3, 117-132.
43. Thakare, R., Singh, A.K., Das, S., Vasudevan, N., Jachak, G.R., Reddy, D.S., Dasgupta, A., Chopra, S., 2017. Repurposing Ivacaftor for treatment of Staphylococcus aureus infections. Int J Antimicrob Ag 50, 389-392.
44. Velkov, T., Abdul Rahim, N., Zhou, Q., Chan, H.-K., Li, J., 2015. Inhaled anti-infective chemotherapy for respiratory tract infections: Successes, challenges and the road ahead. Advanced Drug Delivery Reviews 85, 65-82.
45. Wang, L., Zhang, Y., Tang, X., 2009. Characterization of a new inhalable thymopentin formulation. Int J Pharm 375, 1-7.
46. Wang, S.N., Yu, S.H., Lin, Y.W., Zou, P.Z., Chai, G.H., Ling, J.H., Li, J., Zhou, Q., 2018. Co-Delivery of Ciprofloxacin and Colistin in Liposomal Formulations with Enhanced In Vitro Antimicrobial Activities against Multidrug Resistant Pseudomonas aeruginosa. Pharm Res-Dordr 35.
47. Wang, W.B., Zhou, Q.T., Sun, S.P., Denman, J.A., Gengenbach, T.R., Barraud, N., Rice, S.A., Li, J., Yang, M.S., Chan, H.K., 2016. Effects of Surface Composition on the Aerosolisation and Dissolution of Inhaled Antibiotic Combination Powders Consisting of Colistin and Rifampicin. Aaps J 18, 372-384.
48. Xuejuan Zhang, Z.Z., Yingtong Cui, Fei Liu, Zhengwei Huang, Ying Huang, Rui Zhang, Tim Freeman, Xiangyun Lu, Xin Pan, Wen Tan, Chuanbin Wu, 2018. Effect of powder properties on the aerosolization performance of nanoporous mannitol particles as dry powder inhalation carriers. Powder Technol, 1-9.
49. Zhao, Z.Y., Huang, Z.W., Zhang, X.J., Huang, Y., Cui, Y.T., Ma, C., Wang, G.L., Freeman, T., Lu, X.Y., Pan, X., Wu, C.B., 2018. Low density, good flowability cyclodextrin-raffinose binary carrier for dry powder inhaler: anti-hygroscopicity and aerosolization performance enhancement. Expert Opin Drug Del 15, 443-457.

1. A process for manufacturing a microparticle composite of a polymyxin and a Cystic Fibrosis (CF) transmembrane conductance regulator (CFTR) activator or a CFTR potentiator comprising the steps
a. preparing a suspension of nanoparticles (NPs) of a CFTR activator or a CFTR potentiator;
b. mixing the suspension of NPs with a solution of a polymyxin to afford a mixture suspension;
c. adding the mixture suspension of NPs and the polymyxin to a solution of leucine to afford a solution/suspension of NPs, the polymyxin, and leucine; and
d. spraying freeze dry, spraying dry, or freeze drying the solution/suspension of NPs, the polymyxin, and leucine to afford said micro-particle composite.

2. (canceled)

3. (canceled)

4. (canceled)

5. The process according to claim 1, wherein said polymyxin is polymyxin B, colistin, or a pharmaceutically acceptable salt thereof.

6. The process according to claim 1, wherein said CFTR activator or CFTR potentiator is ivacaftor, elexacaftor, tezacaftor, lumacaftor, or a pharmaceutically acceptable salt thereof.

7. The process according to claim 1, wherein said leucine is L-leucine, D-leucine, an oligomer of L-leucine or D-leucine, or a pharmaceutically acceptable salt thereof.

8. (canceled)

9. (canceled)

10. A pharmaceutical composition comprising the microparticle composite manufactured according to the process of claim 1, together with one or more pharmaceutically acceptable excipients.

11. (canceled)

12. A method for treating a patient with cystic fibrosis (CF) or a lung infection, which method comprises the step of administrating a therapeutically effective amount of a product manufactured according to claim 1 to the patient in need of relief from said CF or lung infection.

13. A microparticle composite or a dry powder inhaler comprising a polymyxin and a CFTR activator or a CFTR potentiator manufactured according to the steps of
a. preparing a suspension of nanoparticles (NPs) of said CFTR activator or said CFTR potentiator;
b. mixing the suspension of NPs with a solution of said polymyxin to afford a mixture suspension;
c. adding the mixture suspension of NPs and said polymyxin to a solution of leucine to afford a solution/suspension of NPs, said polymyxin, and leucine;
d. spraying freeze dry, spraying dry or freeze drying said solution/suspension to afford said micro-particle composite.

14. (canceled)

15. (canceled)

16. (canceled)

17. The microparticle composite or dry powder inhaler according to claim 13, wherein said polymyxin is polymyxin B, colistin, or a pharmaceutically acceptable salt thereof.

18. The microparticle composite or dry powder inhaler according to claim 13, wherein said CFTR activator or CFTR potentiator is ivacaftor, elexacaftor, tezacaftor, lumacaftor, or a pharmaceutically acceptable salt thereof.

19. The microparticle composite or dry powder inhaler according to claim 13, wherein said leucine is L-leucine, D-leucine, an oligomer of L-leucine or D-leucine, or a pharmaceutically acceptable salt thereof.

20. (canceled)

21. (canceled)

22. (canceled)

23. (canceled)

24. The method of claim 12, wherein said polymyxin is polymyxin B, colistin, or a pharmaceutically acceptable salt thereof.

25. The method of claim 12, wherein said CFTR activator or CFTR potentiator is ivacaftor, elexacaftor, tezacaftor, lumacaftor, or a pharmaceutically acceptable salt thereof.

26. The method of claim 12, wherein said leucine is L-leucine, D-leucine, an oligomer of L-leucine or D-leucine, or a pharmaceutically acceptable salt thereof.
